(19) United States
(12) Patent Application Publication
(10) Pub. No.: US 2017/0128173 A1
(43) Pub. Date: May 11, 2017

(54) SCANNING DEVICE

(71) Applicant: Dentsply Sirona Inc., York, PA (US)

(72) Inventors: Markus BERNER, Bülach (CH); Frank THIEL, Ober-Ramstadt (DE)

(73) Assignee: DENTSPLY SIRONA INC., York, PA (US)

(21) Appl. No.: 15/129,728

(22) PCT Filed: Mar. 27, 2015
(86) PCT No.: PCT/EP2015/056694

§ 371 Date: Sep. 27, 2016

(30) Foreign Application Priority Data
Mar. 27, 2014 (DE) 10 2014 205 784.1

(51) Int. Cl. A61C 9/00; A61B 5/00

(52) U.S. Cl. CPC A61C 9/0053; A61B 5/0062

(57) ABSTRACT

The invention relates to a scanning device, comprising a handle (10) to be held by an operating person and a mouthpiece (20). The scanning device is inserted into the mouth of a patient by means of the mouthpiece (20). There, during a recording by means of a sensing apparatus provided in the mouthpiece and a scanning apparatus (60), a section of a dental arch in the mouth is scanned and three-dimensionally measured, wherein many individual scans of different sections of the dental arch are recorded and are combined into a total image. The scanning device also has a drive apparatus (80), which drives the sensing apparatus in such a way that the sensing apparatus moves from one section to a next section of the dental arch in order to perform a particular individual scan, and a control apparatus (70), which controls the drive apparatus (80) in such a way that the drive apparatus moves the sensing apparatus to a desired location of the dental arch.

The invention relates to a scanning device. In particular, the invention relates to a full-jaw scanner.

There is a desire to obtain digital three-dimensional data or 3D data of the teeth or jaw of patients without having to make a silicone impression.

In the prior art, there are scanners, such as the “CEREC OmniCam,” which are inserted into the patient's mouth and then in a single recording are able to measure three-dimensionally only a small section of a dental arch. The scanner must then be guided around manually in the oral cavity in order to make a plurality of individual recordings which are then combined into a total image. In the process, the user is responsible for ensuring that the entire jaw is recorded fully and without omissions. This is done by making a visual inspection of the result and then manually positioning the scanner at a location which is still to be recorded.

In scanning of this kind, there are problems relating to the precision of scanning due to error propagation during the process of combining into a total image. Furthermore, manually guiding the scanner around within the oral cavity is time-consuming and not easy enough that a user can do so without previous training. The visual inspection for completeness and the subsequent manual positioning of the device at the location to be measured are also not easy, which means there is a risk of incomplete data.

There are also scanners, such as the “iTray,” where a patient must bite on a flexible mass which is then measured directly in the mouth.

On the one hand, the handling of a flexible mass is problematic with this type of scanning. On the other, it requires a plurality of sensors.

DE 10 2008 047 816 B4 shows a 3D scanner for scanning teeth which has a carrier and a handle, with projectors and a plurality of cameras mounted on the carrier. In addition, calibration of the cameras is specified and a “registration” or “matching,” that is a combining of individual recordings, is described.

WO 2013/121605 A1 and DE 11 2012 005 886 T5 disclose a device for recording tooth rows in oral cavities, wherein multiple cameras are provided for (concurrently) recording teeth.

US 2002/0058229 A1 discloses the provision of transparent material at a mouthpiece of a device for imaging teeth.

U.S. Pat. No. 6,386,867 B1 discloses a method for scanning teeth in individual scans, which includes a check as to whether everything has been scanned, and a subsequent generation of a 3D model.

U.S. Pat. No. 6,821,116 B1 also discloses a method for scanning teeth.

DE 10 2009 038 588 A1 discloses a method for obtaining from individual data records (with correction) an overall data record of an object to be measured.

DE 38 10 455 A1 discloses a method and a device for the contactless spatial recording of an irregular body, wherein a drive unit for driving recording units can be controlled by an evaluation unit.

The closest pertinent document DE 42 18 219 C2 discloses a device for the contactless measurement of, for example, dental objects, wherein a drive apparatus is provided for driving a sensing apparatus for performing an individual scan and a control apparatus is provided for controlling the drive apparatus for moving the sensing apparatus to a particular location.

It is the task of the present invention to provide a full-jaw scanner and an associated method, wherein scanning is performed in a simple manner and 3D data for an entire jaw are obtained at a high level of precision.

This task is solved by a scanning device as specified in claim 1 and by a scanning method as specified in claim 16. The corresponding dependent claims 2 to 15 and 17 to 23 disclose advantageous developments of the scanning device in claim 1 and of the scanning method in claim 16.

A scanning device is provided, which has a handle to be held by an operating person and a mouthpiece to be inserted into the mouth of a patient, wherein the scanning device comprises a sensing apparatus provided in the mouthpiece and a scanning apparatus, wherein when the mouthpiece is inserted in the mouth of a patient, the sensing apparatus in conjunction with the scanning apparatus during a recording scans and measures three-dimensionally a section of a dental arch inside the mouth, wherein many individual scans of different sections of the dental arch are recorded and combined into a total image.

The scanning device also comprises the following: a drive apparatus which drives the sensing apparatus in such a way that it moves from one section to a next section of the dental arch in order to carry out the corresponding individual scan, and a control apparatus which controls the drive apparatus in such a way that it moves the sensing apparatus to a desired location on the dental arch, wherein the sensing apparatus preferably has at least one mirror which, motor-driven in the mouthpiece by the drive apparatus, is moved in such a way that a scan area is deflected by it to a desired location on the dental arch.

In particular, a jacket is provided for the mouthpiece which is at least partially transparent, wherein the jacket of the mouthpiece or the transparent mouthpiece is provided with precise calibration marks, which are scanned together with a particular individual scan.

The scanning device with movable scanning apparatus makes it advantageously possible to scan an entire jaw without the entire scanning device having to be repositioned within the mouth of a patient by an operating person.

The at least one mirror preferably has at least three degrees of freedom, whereby lingual and/or occlusal and/or buccal scanning by means of the scanning apparatus within the mouth of a patient is made possible.

Preferably, the control apparatus also has a calculation and evaluation device for combining the individual scans to obtain complete data for the jaw while taking into account the scanned calibration marks and thus the position of the mirrors, for analyzing the jaw data obtained in order to determine whether the jaw data are complete, and for completing the jaw data.

The individual scans are for example combined by means of an iterative process for the gradual approximation of two point clouds, which is also called the ICP or iterative closest point method. The approximate position of the mirrors can be used for plausibility checking.

By knowing the position of the calibration marks on the mouthpiece, the overall result can be corrected. It is possible to determine whether measurement is already complete or not—in other words, whether the surface of a dental arch has been fully recorded or whether there are still gaps in the measured surface. A scanning position for an individual scan is then automatically applied to the location of such gaps. If necessary, such locations are measured at different angles. The measurement result obtained with the individual scan is then combined with the other individual scans.

The scanning apparatus is preferably a 3D scanner which can scan over a large depth range, such as 100 mm. With a scanning apparatus of this kind, good images can be recorded.

Due to the motorized drive not only the position but also the viewing direction of the individual scan can advantageously be freely selected and positioned. Since the mirrors have a motorized drive, it is no longer necessary for the scanning device to be moved manually within the mouth of a patient, thereby providing great relief not only for the patient but also for a person operating the scanning device. The motorized drive can also improve accuracy during scanning.

A mirror can also be provided as a movable sensing apparatus, wherein the scanning apparatus is also arranged movably. Alternatively, two mirrors are preferably provided as movable sensing apparatuses, wherein the scanning apparatus is arranged in a fixed position. The latter possibility is to be preferred since mirrors are easier to move than a scanning apparatus.

The drive apparatus is preferably arranged outside the mouthpiece, in other words, in the handle of the scanning device. This arrangement leaves enough free space in the mouthpiece for movement of the sensing apparatus.

The mouthpiece has a top and a bottom face, which are connected by a circumferential side wall. The side wall is provided with an opening toward the handle, through which the mouthpiece transitions into the handle. As scanning device, the handle and mouthpiece form a single unit. The technical apparatuses provided in them are arranged separately so as not to disturb movement of the sensing apparatus. The drive apparatus and the control apparatus are thus not accommodated in the mouthpiece. The control apparatus is preferably accommodated even outside the handle but naturally connected to the technical apparatuses of the scanning device.

Furthermore, the mouthpiece seen from above has the shape of an isosceles trapezoid with one base line which has a width equal to the width of the handle, and an additional base line having a width of about 55 to 85 mm, which is larger than the width of the one base line, wherein the one base line is at a distance of about 45 to 55 mm from the other base line. In addition, the mouthpiece seen from the side has a height of about 20 mm, thereby allowing it to be inserted between the teeth into a patient's mouth.

Jackets of different sizes will preferably be available or the jacket be designed such that it can be adjusted or unfolded. Sets of teeth of different sizes can thus be scanned advantageously. The jacket is thus advantageously removable and can be cleaned and/or sterilized.

Preferably, the mouthpiece can also be heated, wherein the heating is realized by a stream of warm air inside the mouthpiece. In this way, condensation in the mouthpiece can be prevented.

Particularly preferably, two mirrors are provided in the mouthpiece. Here, a central mirror is attached to one end of a support arm mounted outside the handle in the direction of the longer base line of the trapezoid and in the direction of an extension of the handle of the scanning device and an outer mirror is attached to one end of an additional support arm mounted outside the handle in the direction of the longer base line of the trapezoid and extends along a leg of the trapezoid.

The support arm for the central mirror is mounted rigidly and suitably for a translational movement while the central mirror at the end of the support arm is mounted in a supporting frame, wherein the central mirror is mounted in the supporting frame such that it can rotate about an axis parallel to the faces of the mouthpiece, and the supporting frame itself is mounted at the end of the support arm such that it can rotate about an axis orthogonal to the faces of the mouthpiece. The additional support arm for the outer mirror is suitably mounted for a translational movement at its attachment end for a rotational movement about an axis orthogonal to the faces of the mouthpiece and has in a central position a pivot point at which it can be rotated about an axis orthogonal to the faces of the mouthpiece. In addition, the outer mirror is mounted at the end of the additional support arm rotatably about an axis parallel to the faces of the mouthpiece and about an axis orthogonal to the faces of the mouthpiece. The drive apparatus in each case produces the translational movements and the pivoting or rotational movements.

The central mirror preferably has three degrees of freedom. The outer mirror preferably has at least four degrees of freedom.

With the stationary scanning apparatus and two mirrors which can be moved by the drive apparatus, every point on a dental arch can thus be viewed provided the two mirrors have sufficient freedom of movement, which is provided by the described mounting of the mirrors. The mirrors do not need to be precisely driven. The accuracy of scanning does not depend on a precise knowledge of the mirror position. Only a respective individual scan must be accurate.

The invention also relates to a method for scanning an entire jaw with a scanning device according to the invention, said method preferably comprising the following steps:
Insertion of the mouthpiece of the scanning device into a patient's mouth,
Calibration of the scanning device,
Scanning and three-dimensional measurement of a section of the dental arch in the mouth,
Simultaneous scanning of a calibration mark on the mouthpiece jacket or mouthpiece itself, said mark corresponding to the scan position,
Automatic movement of the mirrors in the mouthpiece into a further scanning position and further scanning of a section of the dental arch in the mouth of a calibration mark on the mouthpiece' jacket or mouthpiece corresponding to the scanning position,
Automatic repetition of the preceding step until all sections of the dental arch in the mouth have been scanned, and
Combination of the individual scans to obtain complete data for the jaw.

According to the invention, there is advantageously an automatic switching from one scanning position to another without an operating person of the scanning device needing to take action. The entire jaw is scanned relatively quickly without the scanning device needing to move inside the mouth of a patient, which is a great relief for a patient in comparison with a scanning device moved manually within the mouth by an operating person.

Preferably, the method also includes the step of analyzing the jaw data obtained to determine whether the jaw data are complete and, in the event of incomplete jaw data and thus of gaps in the jaw data, the steps of automatic movement of the mirrors in the mouthpiece to the scanning positions corresponding to the gaps and of a further scanning of a section of the dental arch in the mouth of a calibration mark on the mouthpiece jacket or mouthpiece corresponding to the scanning position, and the step of combining the individual scans carried out for correction purposes with the existing individual scans.

While combining individual scans together can in the case of freehand scanning result in problems of accuracy, since small errors in the individual scans are cumulative, this problem can be solved in a simple manner by the scanning device and method implemented with the scanning device according to the present invention.

Calibration of the scanning device includes the following steps:
Single or multiple scanning of the inside of the jaw while simultaneously scanning the corresponding calibration marks while the scanning position is deflected to the inside of the jaw solely by means of the central mirror,
Rotation of the central mirror and thus scanning the entire dental arch from the inside together with the corresponding calibration marks,
Combining the individual scans into a total scan of the inside of the jaw, wherein the contours of the teeth are in each case joined together,
Analysis of the positions of the calibration marks and comparison with the desired locations of the positions in order to detect a deviation,
Calculation of a rectification rule from the deviation, specifying how the measured calibration marks must be rectified to bring them in line with the actual calibration marks, and
Application of this rectification rule to the measured dental arch in order to correct the jaw data obtained.

After calibration, the following data for the scans of the top and outer side of the dental arch are appended to the measured dental arch.

Since scanning of the inside of the dental arch is very fast, it can even be carried out several times. In this way, it can be detected whether the calibration marks have shifted with respect to the dental arch during scanning.

An inside surface of the teeth is advantageously scanned using only a single mirror which is centrally located in the middle of the oral cavity and which is tilted in such a way that it deflects the scanning section onto the inside surface of the teeth.

When occlusal surfaces are scanned, the scan area is advantageously directed by a central mirror outwardly onto the second mirror, which is positioned orthogonally over or under the teeth, and then deflected by the second mirror by about 90° onto the occlusal surface.

When scanning the outside of the teeth, the second mirror is advantageously positioned further toward the outside than when scanning occlusal surfaces, and the scan area is then deflected from the outside onto the tooth surfaces.

In the case of a mouthpiece that is transparent above and below, switching a scanning run between the upper jaw and the lower jaw is carried out advantageously by tilting the mirrors.

According to the invention, this delivers the advantage of a fast, straightforward and accurate scanning of an entire jaw.

The specified and other features and details of the invention will become clearer to a person skilled in the art from the following detailed description and the accompanying drawings which illustrate with the aid of an example the features of the present invention and in which

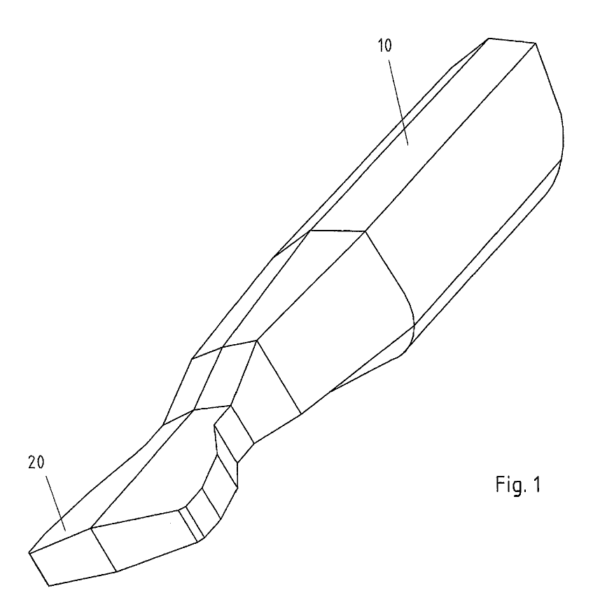

FIG. 1 depicts an oblique view of a scanning device according to the present invention,

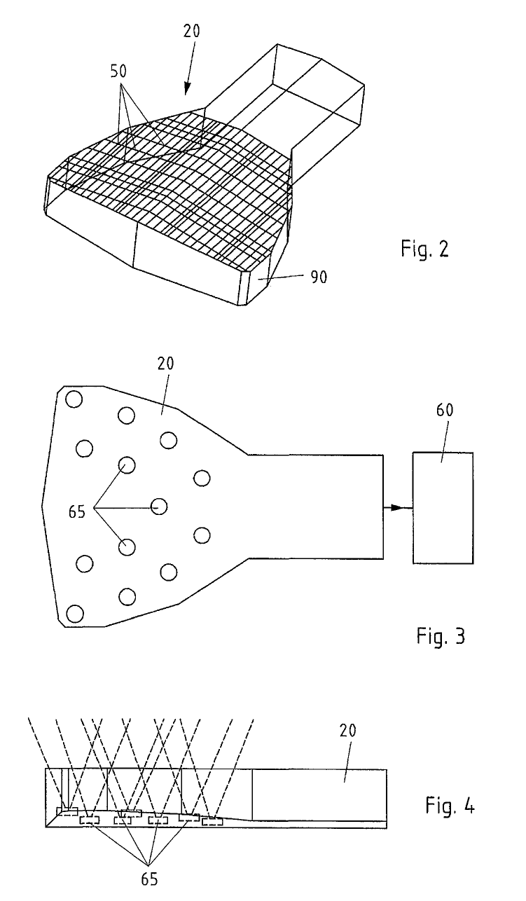

FIG. 2 depicts an oblique view of a mouthpiece with jacket and calibration marks of the scanning device according to the present invention,

FIG. 3 depicts a top view of a mouthpiece of the scanning device with measuring heads according to the present invention,

FIG. 4 depicts a side view of a mouthpiece of the scanning device with measuring heads according to the present invention,

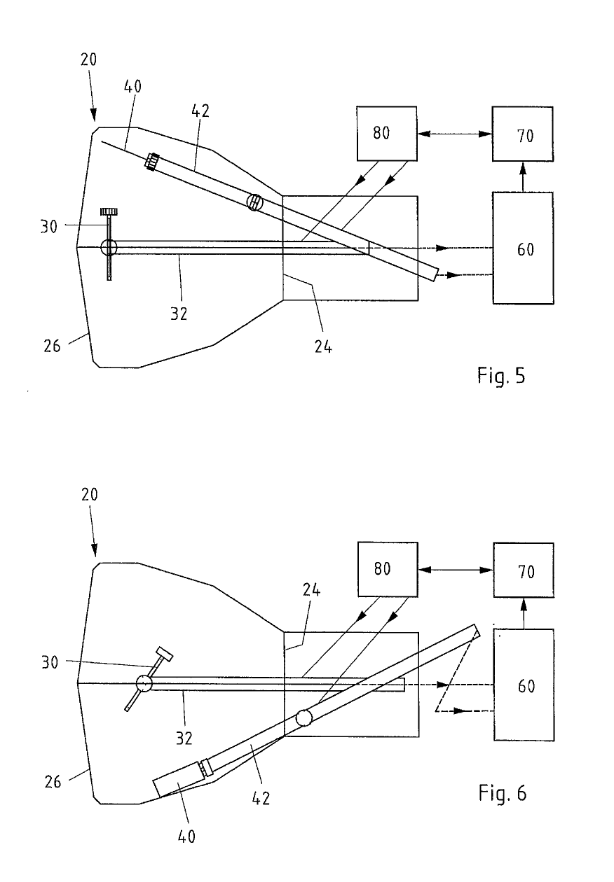

FIG. 5 depicts a top view of the interior of the mouthpiece of the scanning device according to the present invention,

FIG. 6 depicts a further top view of the interior of the mouthpiece of the scanning device according to the present invention,

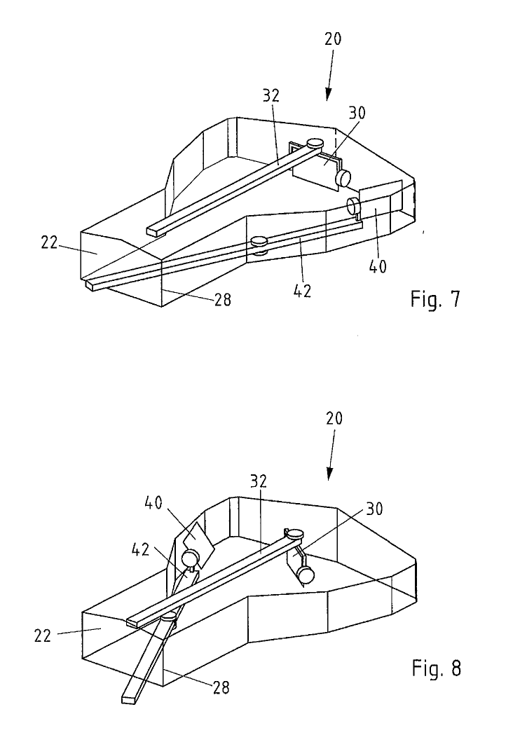

FIG. 7 depicts an oblique view of the open mouthpiece of the scanning device according to the present invention,

FIG. 8 depicts a further oblique view of the open mouthpiece of the scanning device according to the present invention,

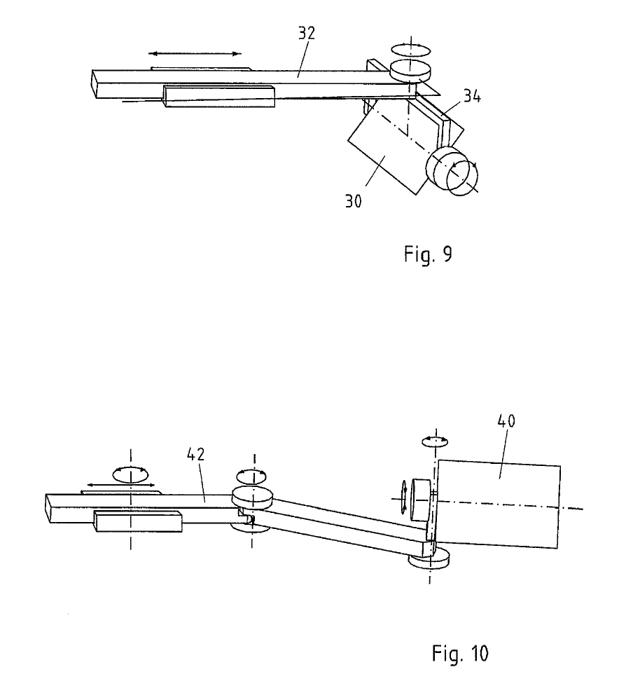

FIG. 9 depicts an oblique view of a central mirror and its support according to the present invention, and

FIG. 10 depicts an oblique view of an outer mirror and its support according to the present invention.

Hereinafter, the present invention will be explained in detail based on preferred embodiments while referring to the figures.

FIG. 1 shows an oblique view of a general structure of a scanning device according to the invention, which shows the handle 10 to be held by an operating person and a mouthpiece 20 connected to this handle 10. The relatively large, at least partially transparent mouthpiece 20 of the scanning device is inserted into the mouth of a patient. Here, during a recording by a scanning apparatus arranged inside the handle 10, a section of a dental arch inside the mouth is scanned in each case and measured three-dimensionally. The scanning apparatus is preferably a camera or a 3D scanner which can scan over a large depth range, such as 100 mm. It can function on the principle of confocal microscopy.

As shown in FIG. 2, the transparent mouthpiece 20 is surrounded by a jacket 90 to which precise calibration marks 50 have been applied, which are scanned together with each individual scan. In this case, the calibration marks 50 are shown with a squared pattern but could also have a different pattern.

Many individual scans are made of different sections of the dental arch and combined into a total image. According to the invention, technical apparatuses are provided in the mouthpiece 20 which under automatic control carry out individual scans of the various sections of the dental arch either simultaneously or in rapid succession.

FIGS. 3 and 4 show a mouthpiece 20 of a scanning device in a top view and a side view respectively. It can be seen that a plurality of measuring heads 65 are arranged in the trapezoidal mouthpiece 20 in such a way that they run along the legs of the trapezoid in two staggered rows. As a result of this arrangement, the measurement areas overlap, as can be seen in particular in FIG. 4. FIG. 3 also indicates how data acquired by the measuring heads 65 are passed on to a scanning apparatus 60.

According to FIGS. 3 and 4, the plurality of measuring heads 65 for the scanning apparatus 60 is therefore positioned in such a way that the entire upper jaw of a patient can be scanned by them at the same time. In order to scan the entire lower jaw, the scanning device must be turned over. It is also conceivable for measuring heads 65 to be provided in such a way that both the upper jaw and lower jaw of a patient can be scanned simultaneously.

A similar principle is known, for example, for the measurement of motor vehicles. For use in measuring an entire upper and/or lower jaw, this principle must however be modified such that the measuring heads take the form of miniature lenses, such as endoscope lenses, for example. Using a plurality of measuring heads makes it possible to scan an entire jaw of a patient very quickly and simultaneously.

As an alternative or addition, a drive apparatus 80 and a control apparatus 70 can also according to the invention be provided in addition to a scanning apparatus 60, as shown in FIG. 5. The drive apparatus 80 drives a sensing apparatus in such a way that it moves from one section to a next section of the dental arch in order to carry out the corresponding individual scan, and the control apparatus 70 controls the drive apparatus 80 in such a way that it moves the sensing apparatus to a desired location on the dental arch.

As is also shown for example in FIGS. 5 and 6, the sensing apparatus has two mirrors 30, 40, which are motor-driven to move in such a way that a scan area can be deflected by them onto a desired location on the dental arch.

In FIGS. 5 and 6, it can be clearly seen that the mouthpiece 20 has a top face and a bottom face which are connected by a circumferential side wall. The side wall is provided with an opening 22 toward the handle 10 of the scanning device, which is to be held by an operating person, through which opening 22 the mouthpiece 20 transitions into the handle 10.

The mouthpiece 20 seen from above has the shape of an isosceles trapezoid with one base line 24 which has a width equal to the width of the handle 10, and another base line 26 having a width of about 55 to 85 mm, which is larger than the width of the one base line 24, wherein the one base line 24 is at a distance of about 45 to 55 mm from the other base line 26, and wherein the mouthpiece 20 seen from the side has a height 28 of about 20 mm, thereby allowing it to be inserted between the teeth into a patient's mouth. As has already been indicated, the mouthpiece 20 is provided with a jacket 90. Jackets 90 are available in different sizes. The jacket 90 can also be designed such that it can be unfolded, thereby being suitable for insertion into differently sized oral cavities of patients. In addition, the mouthpiece 20 can be heated, wherein the heating is realized by a stream of warm air inside the mouthpiece 20.

In FIGS. 5 to 8, it can be clearly seen that two mirrors 30, 40 are provided in the mouthpiece 20. A central mirror 30 is attached to one end of a support arm 32 in the direction of the longer base line 26 of the trapezoid and in the direction of an extension of the handle 10 of the scanning device. An outer mirror 40 is attached to one end of a support arm 42 in the direction of the longer base line 26 of the trapezoid and extends along one leg of the trapezoid.

The support arm 32 for the central mirror 30 is rigidly and suitably mounted for a translational movement while the central mirror 30 at the end of the support arm 32 is mounted in a supporting frame 34, wherein the central mirror 30 is mounted in the supporting frame 34 such that it can rotate about an axis parallel to the faces of the mouthpiece 20, and the supporting frame 34 itself is mounted at the end of the support arm 32 such that it can rotate about an axis orthogonal to the faces of the mouthpiece 20. The additional support arm 42 for the outer mirror 40 is suitably mounted for a translational movement and at its attachment end for a rotational movement about an axis orthogonal to the faces of the mouthpiece 20 and has in a central position a pivot point at which it can be rotated about an axis orthogonal to the faces of the mouthpiece 20, and the outer mirror 40 is mounted at the end of the additional support arm 42 rotatably about an axis parallel to the faces of the mouthpiece 20 and about an axis orthogonal to the faces of the mouthpiece 20. The drive apparatus 80 produces the translational movements and the pivoting or rotational movements.

Now that the mouthpiece 20 has been described according to a preferred embodiment of the present invention together with the mirrors 30, 40 and their supports in the mouthpiece, FIGS. 5 to 10 will be briefly addressed.

In each case, FIGS. 5 and 6 show a top view of a mouthpiece 20 with support arms 32 and 42, on which a central mirror 30 and an outer mirror 40 are mounted respectively.

The two figures show the support arms 32, 42 and the mirrors 30, 40 in different positions.

In each case, FIGS. 7 and 8 show an oblique view of a mouthpiece 20 with support arms 32 and 42, on which a central mirror 30 and an outer mirror 40 are mounted respectively. The two figures, corresponding to FIGS. 3 and 4, each show a different position of the support arms 32, 42 and mirrors 30, 40.

Finally, FIGS. 9 and 10 show respectively a support arm 32 with the central mirror 30 and a support arm 42 with the outer mirror 40. The aforementioned movable and rotatable mounting of the mirrors 30, 40 can be seen clearly in these drawings.

Generally speaking, space inside the mouthpiece 20 is tight. For this reason, most of the drive elements are preferably arranged outside the mouthpiece 20. Drives or drive elements accommodated inside the mouthpiece 20 must be very compact.

In the present description, the mirror 30 and the mirror 40 have different designs. The mirror 30 can have a simpler design than the mirror 40. It can however also be advantageous for both mirrors to have the same design, in which case they would both have the same design as the mirror 40 which has been described. This mirror is able to do everything that the mirror 30 can.

In particular, it can be seen from FIG. 9 that the mirror 30 has three degrees of freedom. There are a translational axis and two rotational axes. The support arm 32 is guided in a linear guide and can be moved in a motorized manner. This linear movement can be effected, for example, by a spindle drive or a linear motor. The mounting and drive of this support arm 32 are preferably arranged outside the mouthpiece 20. One rotational axis relates to the supporting frame 34. The supporting frame 34 attached to the support arm 32 can be rotated. It is mounted, for example, with a miniature ball bearing. It can be driven, for example, from one side, such as is shown in FIG. 9. A reset can be obtained by means of a return spring. Another possibility would be a miniature motor, such as, for example, an ultrasonic drive. A miniature motor of this kind could be accommodated in a space for the drive that is sketched in a disk-like manner. In the case of a solution with side, the drive for this axis can be arranged outside the mouthpiece 20. The mirror 30 only needs to be tilted about a further rotational axis. This advantageously takes the form of a miniature motor or a magnetic drive, such as in a moving-coil instrument.

In addition, it can be seen in particular from FIG. 10 that the mirror 40 has five degrees of freedom. Fewer degrees of freedom are possible, but then certain parts of a patient's mouth would be difficult to view. Given the current state of knowledge at least four degrees of freedom are necessary.

As with the mirror 30, there is a translational axis. Furthermore, the translational axis has a rotational axis. Linear guide and drive are arranged rotatably. The mounting and drive can preferably be arranged outside the mouthpiece. A miniature motor is used for the drive. There is then a rotational axis identical to the other rotational axis of the mirror 30. The other rotational axes have drives like the first rotational axis of the mirror 30.

It should also be mentioned that the control apparatus also has a calculation and evaluation device for combining the individual scans to obtain complete data for the jaw while taking into account the scanned calibration marks and thus the position of the mirrors, for analyzing the jaw data obtained in order to determine whether the jaw data are complete, and for completing the jaw data.

Since a preferred embodiment of a scanning device according to the present invention has been described in detail above, a description now follows of a scanning method carried out with this scanning device.

First of all, the mouthpiece 20 of the scanning device is inserted into the mouth of a patient in order to then calibrate the scanning device. A section of a dental arch inside the mouth is then scanned and measured three-dimensionally while at the same time a calibration mark 50 on the mouthpiece 20 corresponding to the scan position is scanned. The mirror or the mirrors 30, 40 in the mouthpiece 20 are then moved automatically to another scanning position and another section of the dental arch in the mouth of a calibration mark 50 on the mouthpiece 20 corresponding to the scanning position is scanned. This step is repeated automatically until all sections of the dental arch in the mouth have been scanned. The individual scans are then combined so as to obtain the complete data for the jaw.

To switch between the lower and the upper jaw, the scanning device can be removed from the mouth, rotated by 180° and then reinserted. It is however also possible to switch between the lower and the upper jaw by tilting the two mirrors 30 and 40 appropriately. The mirrors 30 and 40 can thus direct the light downwardly instead of upwardly. In this way, both the upper and the lower jaw can be scanned without temporarily taking the scanning device out of the mouth. In this case, the mouthpiece must be transparent at the top face and the bottom face and have calibration marks on both sides.

The inside surface (lingual) can be scanned advantageously by using only one mirror. To do so, this mirror 30 is centrally located in the middle of the oral cavity and is tilted in such a way that it directs the scanning section onto the inside surface of the teeth.

To scan the occlusal surfaces, the scan area is first deflected by a central mirror 30 outwardly onto the second mirror 40, which is positioned orthogonally over or under the teeth, and then deflected by this second mirror 40 by about 90° onto the occlusal surface.

To scan the outer side (buccal), the procedure is similar to that for occlusal surfaces. Here, however, the second mirror 40 is positioned further outwardly and the scan area then deflected from outside onto the tooth surface.

This outer position of the second mirror 40 is its outermost position—it determines the minimum size of the mouthpiece.

At each mirror, the scan area is of course mirrored—in other words, it is laterally reversed. The data obtained from the scanning are thus initially acquired with lateral reversal. With two mirrors 30 and 40, a scan is restored to normal. When evaluating, it is therefore necessary to know how many mirrors were used in an individual scan and a corresponding reverse mirroring must then be carried out. In principle, any sequence can be selected for a scanning run. Following a preferred sequence, the inside is scanned first, then the occlusal surfaces and finally the outside.

The jaw data so obtained are analyzed to ascertain whether the jaw data are complete. In the event of incomplete jaw data and thus of gaps in the jaw data, the mirror or the mirrors 30, 40 in the mouthpiece 20 are moved automatically to the scanning positions corresponding to the gaps. There is a further scan of a section of the dental arch in the mouth of a calibration mark 50 on the mouthpiece 20 corresponding to the scanning position and finally the individual scans carried out for correction purposes are combined with the existing individual scans.

Calibration is carried out by single or multiple scanning of the inside of the jaw while simultaneously scanning the corresponding calibration marks 50 while the scanning position is deflected to the inside of the jaw solely by means of the central mirror 30, by rotation of the central mirror 30 and thus scanning the entire dental arch from the inside together with the corresponding calibration marks 50, by combining the individual scans into a total scan of the inside of the jaw, wherein the contours of the teeth are in each case joined together, by analyzing the positions of the calibration marks 50 and comparing them with the desired locations of the positions in order to detect a deviation, by calculating a rectification rule from the deviation, specifying how the measured calibration marks 50 must be rectified to bring them in line with the actual calibration marks 50, and by applying this rectification rule to the measured dental arch in order to correct the jaw data obtained. After calibration, the subsequent data received for the scans of the top and outside of the dental arch are appended to the measured dental arch.

The present invention discloses a scanning device and a scanning method, in which an operating person only needs to place the scanning device in the mouth of a patient once, after which the scanning process runs automatically.

An entire upper or lower jaw can be fully measured without a scanning apparatus needing to be moved relative to the jaw. This means that an operating person does not have to follow a special measurement strategy for measurement to be carried out without omissions.

In simple terms, the concept according to the present invention is that a 3D measurement camera arranged outside the oral cavity serves as a scanning apparatus with a measuring extension for the oral cavity. With a differently designed measurement arrangement, the 3D measurement camera could in principle measure other geometries as well. The measuring extension thus represents an extension which has such a flexible design that a large number of measurement perspectives can be acquired. In comparison with known systems, such as, for example, the “CEREC Omnicam” which was mentioned at the beginning, this mirror system replaces manual movement. It is also conceivable, instead of automatically movable mirrors, for a plurality of measuring heads to be provided in the mouthpiece of the scanning device, positioned there in such a way that an entire jaw can be recorded. It is also possible to provide a scanning device with automatically movable mirrors and multiple measuring heads.

1. A scanning device with a handle to be held by an operating person and a mouthpiece to be inserted into the mouth of a patient, wherein the scanning device has a sensing apparatus provided in the mouthpiece and a scanning apparatus, wherein when the mouthpiece is inserted in the mouth of a patient, the sensing apparatus in conjunction with the scanning apparatus during a recording in each case scans and measures three-dimensionally a section of a dental arch inside the mouth, wherein many individual scans of different sections of the dental arch are recorded and combined into a total image, wherein the scanning device further comprises:
a drive apparatus which drives the sensing apparatus in such a way that it moves from one section to a next section of the dental arch in order to carry out a corresponding individual scan, and
a control apparatus which controls the drive apparatus in such a way that it moves the sensing apparatus to a desired location on the dental arch,
wherein the sensing apparatus preferably has at least one mirror which, motor-driven in the mouthpiece by the drive apparatus, is moved in such a way that a scan area is deflected by the mirror to a desired location on the dental arch,
wherein a jacket, being provided for the mouthpiece, which is at least partially transparent, and
wherein the jacket of the mouthpiece is provided with precise calibration marks which are scanned together with a particular individual scan.

2. The scanning device according to claim 1, characterized by wherein the at least one mirror has at least three degrees of freedom, whereby lingual and/or occlusal and/or buccal scanning within the mouth of a patient is made possible by means of the scanning apparatus.

3. The scanning apparatus according to claim 1, wherein the control apparatus has a calculation and evaluation device for combining the individual scans to obtain complete data for the jaw while taking into account the scanned calibration markers and thus the corresponding position of the mirror or the mirrors, for analyzing the jaw data obtained in order to determine whether the jaw data are complete, and for completing the jaw data.

4. The scanning device according to claim 1, wherein the scanning apparatus being is a 3D scanner which scans over a large depth range.

5. The scanning device according to claim 1, is provided with a mirror and the scanning apparatus being movably arranged.

6. The scanning device according to claim 1, is provided with two mirrors and the scanning apparatus being immovably arranged.

7. The scanning device according to claim 1, wherein the drive apparatus being arranged outside the mouthpiece.

8. The scanning device according to claim 1, wherein the mouthpiece having a top face and a bottom face which are connected by a circumferential side wall, wherein the side wall is provided with an opening toward the handle of the scanning device, through which opening the mouthpiece transitions into the handle.

9. The scanning device according to claim 1, wherein the mouthpiece seen from above having the shape of an isosceles trapezoid with one base line which has a width equal to the width of the handle, and another base line having a width of about 55 to 85 mm, which is larger than the width of the one base line, wherein the one base line is at a distance of about 45 to 55 mm from the other base line, and wherein the mouthpiece seen from the side has a height, thereby allowing it to be inserted between the teeth and into a patient's mouth.

10. The scanning device according to claim 1, wherein jackets of different sizes being provided or the jacket being designed such that it can be adjusted or unfolded.

11. The scanning device according to claim 1, wherein the mouthpiece being heatable, wherein the heating is realized by a stream of warm air inside the mouthpiece.

12. The scanning device according to claim 6, wherein the two mirrors having a central mirror attached to one end of a rigid support arm, which is mounted outside the handle in the direction of the longer base line of the trapezoid and in the direction of an extension of the handle of the scanning device, and an outer mirror attached to one end of an additioinal support arm, which is mounted outside the handle, in the direction of the longer base line of the trapezoid and extending along one leg of the trapezoid.

13. The scanning device according to claim 12, wherein the support arm for the central mirror being rigidly and suitably mounted for a translational movement and the central mirror at the end of the support arm being mounted in a supporting frame, wherein the central mirror is mounted in the supporting frame such that it can rotate about an axis parallel to the faces of the mouthpiece, and the supporting frame itself is mounted at the end of the support arm such that it can rotate about an axis orthogonal to the faces of the mouthpiece, and the additional support arm for the outer mirror being suitably mounted for a translational movement and at its attachment end for a rotational movement about an axis orthogonal to the faces of the mouthpiece and having in a central position a pivot point, at which it can be rotated about an axis orthogonal to the faces of the mouthpiece , and the outer mirror being mounted at the end of the additional support arm rotatably about an axis parallel to the faces of the mouthpiece and about an axis orthogonal to the faces of the mouthpiece, wherein the drive apparatus produces the translational movements and the pivoting or rotational movements.

14. The scanning device according to claim 12, characterized by wherein the central mirror having three degrees of freedom.

15. The scanning device according to claim 12, wherein the outer mirror having at least four degrees of freedom.

16. A method for scanning an entire jaw with a scanning device according to claim 1, the method comprising the following steps:
insertion of the mouthpiece of the scanning device into a patient's mouth,
calibration of the scanning device,
scanning and three-dimensional measurement of a section of the dental arch in the mouth by means of the scanning device,
simultaneous scanning of a calibration mark on the jacket of the mouthpiece, said mark corresponding to the scan position,
automatic movement of the mirror or the mirrors in the mouthpiece into a further scanning position and further scanning of a section of the dental arch in the mouth of a calibration mark on the jacket of the mouthpiece corresponding to the scanning position,
automatic repetition of the preceding step until all sections of the dental arch in the mouth have been scanned, and
combination of the individual scans to obtain complete data for the jaw.

17. The method according to claim 16, comprising in addition the following steps:
analysis of the jaw data obtained in order to determine whether the jaw data are complete, and
in the case of incomplete jaw data and thus of gaps in the jaw data,
corresponding automatic movement of the mirror or the mirrors in the mouthpiece into the scanning positions corresponding to the gaps,
further scanning of a section of the dental arch in the mouth of a calibration mark on the jacket of the mouthpiece corresponding to the scanning position, and
combination of the individual scans carried out for corrective purposes with the individual scans already carried out.

18. The method according to claim 16, wherein the calibration comprising the following steps:
single or multiple scanning of the inside of the jaw while simultaneously scanning the corresponding calibration marks while the scanning position is deflected to the inside of the jaw solely by means of the central mirror,
rotation of the central mirror and thus scanning the entire dental arch from the inside together with the corresponding calibration marks,
combining the individual scans into a total scan of the inside of the jaw, wherein the contours of the teeth are in each case joined together,
analysis of the positions of the calibration marks and comparison with the desired locations of the positions in order to detect a deviation,
calculation of a rectification rule from the deviation, specifying how the measured calibration marks must be rectified to bring them in line with the actual calibration marks, and
application of this rectification rule to the measured dental arch in order to correct the jaw data obtained.

19. The method according to claim 18, wherein the following data for the scans of the top and outside of the dental arch being appended to the measured dental arch after calibration.

20. The method according to one of claim 16, further comprising an inside surface of the teeth being scanned using only a single mirror, which is centrally located in the middle of the oral cavity and which is tilted in such a way that it directs the scanning section onto the inside surface of the teeth.

21. The method according to claim 16, wherein the scan area, when occlusal surfaces are scanned, being directed by a central mirror outwardly onto the second mirror, which is positioned orthogonally over or under the teeth, and then deflected by the second mirror by about 90° onto the occlusal surface.

22. The method according to claim 21, wherein the second mirror being positioned further toward the outside when scanning the outside of teeth than when scanning occlusal surfaces, and the scan area then being deflected from the outside onto the tooth surfaces.

23. The method according to claim 16, wherein in the case of a mouthpiece that is transparent above and below, by a scanning run being switched between the upper jaw and the lower jaw by tilting the mirrors.
